(19) United States
(12) Patent Application Publication
(10) Pub. No.: US 2024/0288635 A1
(43) Pub. Date: Aug. 29, 2024

(54) BEAM DIFFRACTION FOR SURGICAL LASER

(71) Applicant: Lumenis Ltd., Yokneam Ilit (IL)

(72) Inventor: Asaf Granot, Lotem (IL)

(73) Assignee: Lumenis Ltd., Yokneam Ilit (IL)

(21) Appl. No.: 18/587,511

(22) Filed: Feb. 26, 2024

Related U.S. Application Data
(60) Provisional application No. 63/448,985, filed on Feb. 28, 2023.

(51) Int. Cl. G02B 6/26; A61B 18/22; A61B 18/26; G02B 6/34

(52) U.S. Cl. CPC G02B 6/262; A61B 18/26; G02B 6/34; A61B 2018/2244; A61B 2018/2261; A61B 2018/2294

(57) ABSTRACT

This disclosure teaches the use of an endoscopic surgical laser with beam diffraction. During a lithotripsy procedure, an endoscopic probe with a laser fiber is deployed. A diffraction grating within the optical core of the laser fiber disperses the laser energy over a larger area for more effective ablation of renal calculi.

### CROSS-REFERENCE TO RELATED APPLICATIONS

This application claims the benefit of U.S. Provisional Patent Application Ser. No. 63/448,985 filed Feb. 28, 2023, the disclosure of which is incorporated herein by reference.

### TECHNICAL FIELD

The present disclosure generally relates to surgical laser system. Particularly, but not exclusively, the present disclosure relates to laser fibers used during endoscopic laser procedures.

### BACKGROUND

Medical lasers are used in a variety of practice areas. One procedure to address renal calculi, also known as kidney stones, is ureteral endoscopy, also known as ureteroscopy. An endoscopic probe, with a camera or other sensor, is inserted into the patient's urinary tract to locate the calculi for removal. In endoscopic lithotripsy, the probe also includes an optical fiber, which conducts a laser beam to disintegrate the calculi as they are found.

An ideal lithotripsy procedure is fast, precise, and thorough: the medical practitioner minimizes the time that the endoscope is inserted, directs all of the discharged laser energy into target calculi, and assures that no large stones or fragments remain. However, these three goals can sometimes be in tension. For larger calculi, a relatively low-intensity but high-frequency pulse may be used to ablate layers from the target without splitting it apart (known as “dusting”). While thorough, this procedure is time-consuming, in part because of the maximum intensity that can be applied without risking premature fragmentation.

A need therefore exists for a device that can facilitate a safe, effective disintegration of targeted calculi more rapidly.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides endoscopic lithotripsy solutions that provide for more efficient disintegration of calculi.

In general, the present disclosure provides for beam diffraction of laser energy used in endoscopic surgical procedures. A grating pattern within the optical core of the laser fiber distributes the beam over a wider area, disintegrating the target more evenly and more quickly.

In some examples, the present disclosure provides an endoscopic surgical device, comprising a laser source a laser fiber optically coupled to the laser source, the laser fiber comprising an optical core and a fiber tip, the optical core comprising a plurality of grating patterns forming a diffraction grating; and an endoscopic probe housing an imager and the fiber tip.

In some implementations, the laser source is a pulsed high-energy laser. In some implementations, the plurality of grating patterns are spaced evenly along a length of the optical core. In some implementations, the grating patterns are spaced at intervals of between 1.9 and 35 microns along the optical core. In some implementations, the intervals are between 7.5 and 7.9 microns. In some implementations, the optical core has a diameter of between 200 and 550 microns. In some implementations, each of the plurality of grating patterns is inscribed on the optical core by mechanical, chemical, or laser etching. In some implementations, each of the plurality of grating patterns is inserted into the optical core by implantation or material compositing. In some implementations, the diffraction grating is disposed within the fiber tip. In some implementations, a distal end surface of the fiber tip is inscribed with one of the plurality of grating patterns.

The device may further comprise a display device configured to display imaging data received from the imager while the endoscopic probe is deployed. The device may further comprise a controller configured to, while the endoscopic probe is deployed, receive imaging data from the imager and activate the laser source to discharge laser energy through the laser fiber, the laser energy emitted from the fiber tip. In some implementations, laser energy is dispersed over an area exceeding ten times the diameter of the laser tip. In some implementations, the laser energy is dispersed over an area that includes a plurality of regions of high intensity separated by regions of lower intensity.

In some embodiments, the present disclosure can be implemented as a method of endoscopic lithotripsy in which any embodiment of the devices described herein is deployed. Laser energy is discharged from the laser source through the laser fiber to ablate a portion of a renal calculus.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an endoscopic surgical system, in accordance with embodiment(s) of the present disclosure.

FIG. 2 illustrates a laser fiber with a diffraction grating, in accordance with embodiment(s) of the present disclosure.

FIG. 3 illustrates an undispersed laser intensity pattern, in accordance with embodiments(s) of the present disclosure.

FIGS. 4A and 4B illustrate dispersed laser intensity patterns, in accordance with embodiment(s) of the present disclosure.

FIG. 5 illustrates a computer-readable storage medium 500 in accordance with one embodiment.

FIG. 6 illustrates a diagrammatic representation of a machine 600 in the form of a computer system within which a set of instructions may be executed for causing the machine to perform any one or more of the methodologies discussed herein, according to an example embodiment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

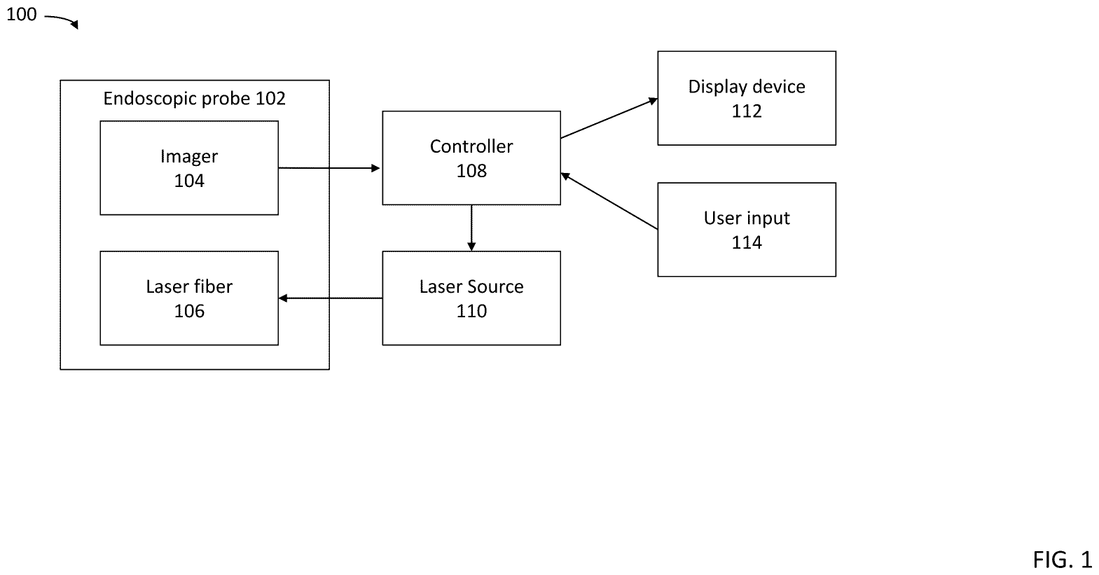

FIG. 1 illustrates an endoscopic surgical system 100, in accordance with non-limiting examples of the present disclosure. In general, an endoscopic surgical system 100 is a system for deploying and controlling an endoscopic probe for imaging, targeting, and destroying calculi. Although the present disclosure focuses of lithotripsy, which would use a ureteroscope, the disclosure is applicable to other urological or endoscopic modalities and/or applications, such as, for example, utilizing duodenoscopes, gastroscopes, colonoscopes, bronchoscopes, etc.

Endoscopic surgical system 100 includes an endoscopic probe 102 that is disposed during lithotripsy. The probe 102 includes an imager 104 and laser fiber 106. The imager 104 can return endoscopic images, video, or other data of the patient's urinary tract while the endoscopic probe 102 is deployed. In some embodiments, imager 104 can be a camera or other sensor deployed with an endoscope during a lithotripsy procedure.

Although the disclosure uses visual-spectrum camera images to describe illustrative embodiments, imager 104 can be any endoscopic imaging device, such as, for example, a fluoroscopy imaging device, an ultrasound imaging device, an infrared or ultraviolet imaging device, a computed tomography (CT) imaging device, a magnetic resonance (MR) imaging device, a positron emission tomography (PET) imaging device, or a single-photon emission computed tomography (SPECT) imaging device.

Imager 104 can generate information elements, or data, including indications of renal calculi. A controller 108 for the surgical system 100 is communicatively coupled to imager 104 and can receive the data including the endoscopic images from imager 104. In general, endoscopic images can include indications of shape data and/or appearance data of the urinary tract. Shape data can include landmarks, surfaces, and boundaries of the three-dimensional surfaces of the urinary tract. With some examples, endoscopic images can be constructed from two-dimensional (2D) or three-dimensional (3D) images.

The laser fiber 106 is optically coupled to a laser source 110. The laser source 110 generates electromagnetic radiation or laser energy in the form of a laser beam in accordance with conventional techniques. Laser energy is discharged through the tip of the laser fiber 106, which is in turn supported by the body of the endoscopic probe 102.

In general, a display device 112 can be a digital display arranged to receive rendered image data and display the data in a graphical user interface. Controller 108 can be any of a variety of computing devices. In some embodiments, controller 108 can be incorporated into and/or implemented by a console of display device 112. With some embodiments, controller 108 can be a workstation or server communicatively coupled to imager 104 and/or display device 112. With still other embodiments, controller 108 can be provided by a cloud-based computing device, such as, by a computing as a service system accessibly over a network (e.g., the Internet, an intranet, a wide area network, or the like). User input 114 may include dedicated components such as a keyboard, and may also be integrated with the display device 112 in the form of capacitive touch controls. In some implementations, remote user input may also be received and used by the controller 108.

It is noted that endoscopic surgical system 100 includes custom components, which are specifically configured, programmed, and/or arranged to carry out the logic flows and methods detailed herein. For example, controller 108 can be preconfigured to execute laser control and discharge as further described.

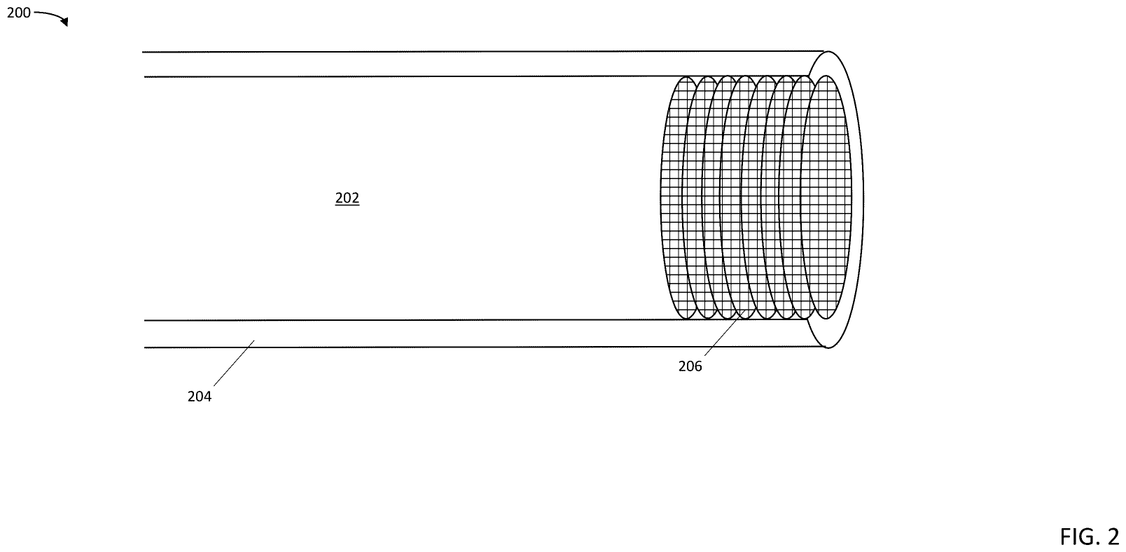

FIG. 2 shows a laser fiber 200 used in conjunction with an endoscopic surgical system as described. The fiber 200 includes an optical core 202 surrounded by a coating 204. The optical core 202 is transparent to the laser wavelengths conducted through the fiber 200, while the coating 204 is reflective of these wavelengths and acts as a waveguide for the laser energy discharged through the fiber 200 during lithotripsy.

The laser fiber 200 includes a series of gratings 206 inscribed within the optical core 200. In some implementations, the gratings 206 are inscribed into the core 200 by a mechanical, chemical, or laser etching process. Compositing, implantation, or another optical engineering method may be used in place of, or in addition to, etching in order to generate the difference in refractive index necessary to affect a dispersion grating. The gratings 206 are spaced along the core at set intervals in order to form a Bragg grating within the optical fiber.

For effective endoscopic lithotripsy, the overall size of the endoscopic probe, and therefore the diameter of the fiber 200, may be limited. For example, the fiber 200 may be limited to an overall diameter of 300 microns. This further limits the available laser wavelengths and possible dispersion patterns.

The size and spacing of the gratings 206 depends on the laser type and settings. For example, a pulsed Holmium laser may project energy at a wavelength of 2100 nm. In this case, a grating size of no more than 0.48 lines per micron is used. Depending on the preferred dispersal pattern, any grating size between 0.03 and 0.48 lines per micron may be chosen (which corresponds to a spacing between lines of between 2.1 and 35 microns). A basic dispersion grating is formed from horizontal and vertical lines etched perpendicular to the longitudinal axis of the fiber, although other patterns could be used. As another example, a pulsed Thulium laser, projecting energy at a wavelength of 1940 nm, may have a grating size of no more than 0.51 lines per micron (corresponding to a spacing of 1.96 microns).

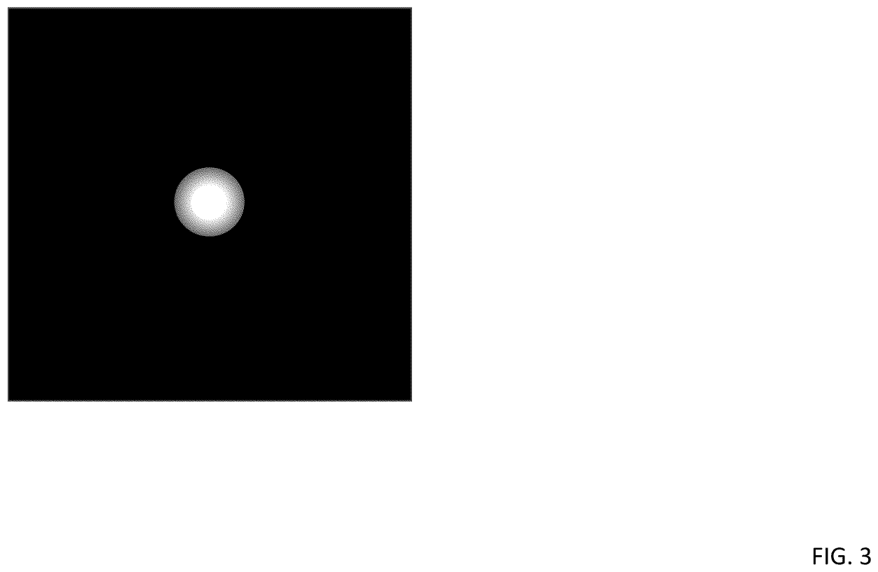

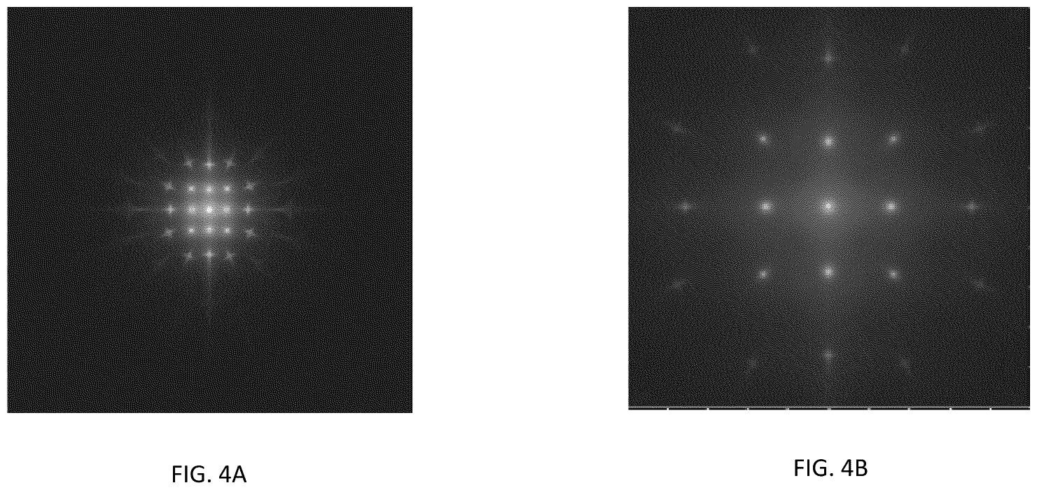

FIG. 3 is a positional graph illustrating a laser pulse emitted from a fiber with no grating as described, while FIGS. 4A and 4B illustrate a laser pulse from a fiber with the inscribed grating. In FIG. 3, the energy of the laser pulse is tightly clustered near the center of the graph. This limits the maximum energy and pulse frequency that can be used without fragmenting a target stone positioned in front of the laser fiber.

In FIG. 4A, which represents the energy intensity 1 mm from a fiber tip as described herein, the energy is dispersed over a larger area due to the grating. Because of this dispersion, more laser energy can be directed at the target without risk of fragmentation. FIG. 4B shows the intensity at 3 mm from the fiber tip; this would allow even more beam dispersion if a greater distance from the target is selected, such as for larger calculi.

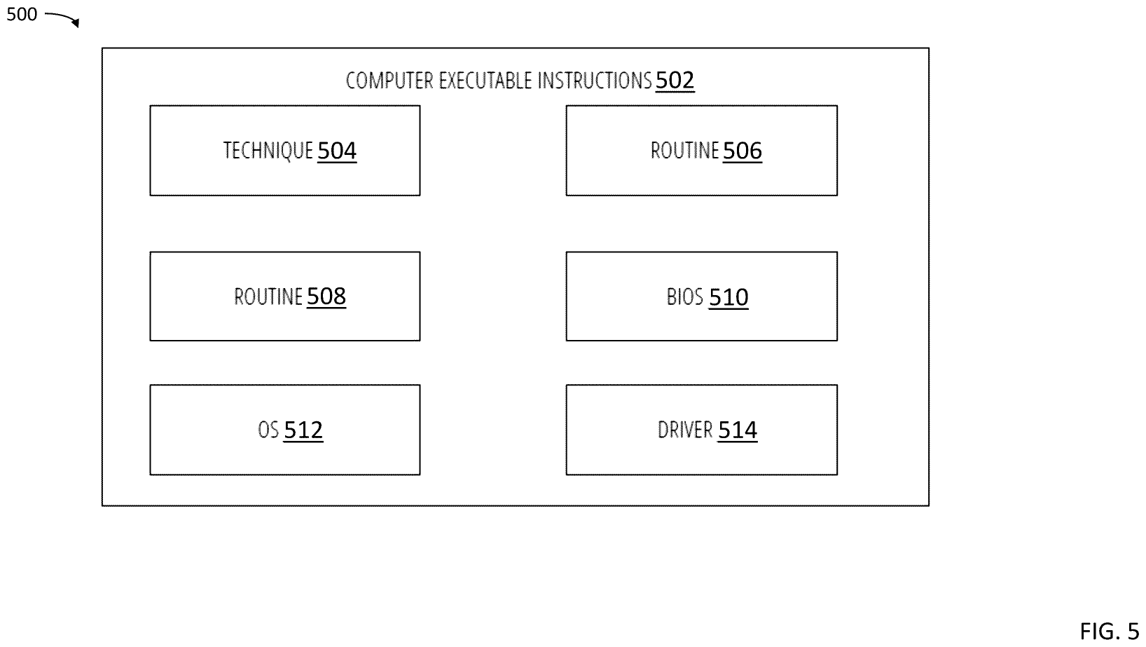

FIG. 5 illustrates computer-readable storage medium 500. Computer-readable storage medium 500 may comprise any non-transitory computer-readable storage medium or machine-readable storage medium, such as an optical, magnetic or semiconductor storage medium. In various embodiments, computer-readable storage medium 500 may comprise an article of manufacture. In some embodiments, the medium 500 may store computer executable instructions 502 with which circuitry (e.g., processor(s) or the like) can execute. For example, computer executable instructions 502 can include instructions to implement operations described with respect to technique 504, routine 506, routine 508, BIOS 510, OS 512, and/or driver 514. Examples of computer-readable storage medium 500 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 502 may include any suitable type of code, such as source code, compiled code, interpreted code, executable code, static code, dynamic code, object-oriented code, visual code, and the like.

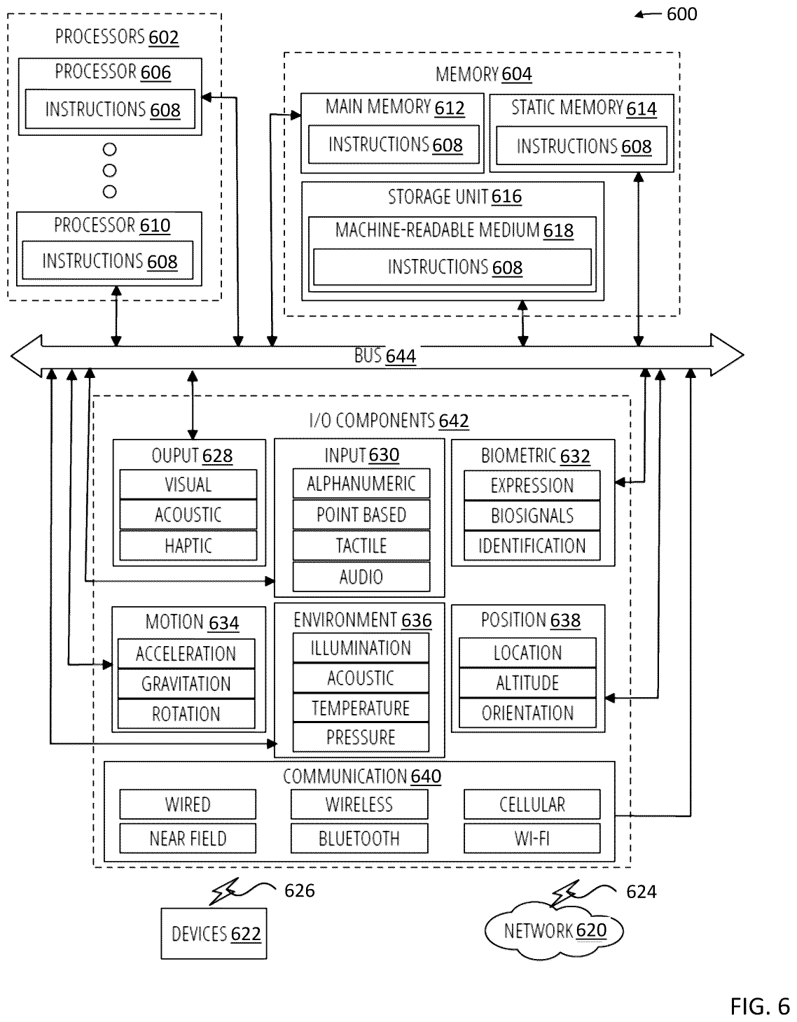

FIG. 6 illustrates a diagrammatic representation of a machine 600 in the form of a computer system within which a set of instructions may be executed for causing the machine to perform any one or more of the methodologies discussed herein. More specifically, FIG. 6 shows a diagrammatic representation of the machine 600 in the example form of a computer system, within which instructions 608 (e.g., software, a program, an application, an applet, an app, or other executable code) for causing the machine 600 to perform any one or more of the methodologies discussed herein may be executed. For example the instructions 608 may cause the machine 600 to execute technique 504 of FIG. 5, routine 506 of FIG. 5, routine 508 of FIG. 5, or the like. More generally, the instructions 608 may cause the machine 600 to allocate memory during pre-boot operations and preserve the memory for post-boot operations (or usage).

The instructions 608 transform the general, non-programmed machine 600 into a particular machine 600 programmed to carry out the described and illustrated functions in a specific manner. In alternative embodiments, the machine 600 operates as a standalone device or may be coupled (e.g., networked) to other machines. In a networked deployment, the machine 600 may operate in the capacity of a server machine or a client machine in a server-client network environment, or as a peer machine in a peer-to-peer (or distributed) network environment. The machine 600 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 608, sequentially or otherwise, that specify actions to be taken by the machine 600. Further, while only a single machine 600 is illustrated, the term “machine” shall also be taken to include a collection of machines 600 that individually or jointly execute the instructions 608 to perform any one or more of the methodologies discussed herein.

The machine 600 may include processors 602, memory 604, and I/O components 642, which may be configured to communicate with each other such as via a bus 644. In an example embodiment, the processors 602 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 606 and a processor 610 that may execute the instructions 608. The term “processor” is intended to include multi-core processors that may comprise two or more independent processors (sometimes referred to as “cores”) that may execute instructions contemporaneously. Although FIG. 6 shows multiple processors 602, the machine 600 may include a single processor with a single core, a single processor with multiple cores (e.g., a multi-core processor), multiple processors with a single core, multiple processors with multiples cores, or any combination thereof.

The memory 604 may include a main memory 612, a static memory 614, and a storage unit 616, both accessible to the processors 602 such as via the bus 644. The main memory 604, the static memory 614, and storage unit 616 store the instructions 608 embodying any one or more of the methodologies or functions described herein. The instructions 608 may also reside, completely or partially, within the main memory 612, within the static memory 614, within machine-readable medium 618 within the storage unit 616, within at least one of the processors 602 (e.g., within the processor's cache memory), or any suitable combination thereof, during execution thereof by the machine 600.

The I/O components 642 may include a wide variety of components to receive input, provide output, produce output, transmit information, exchange information, capture measurements, and so on. The specific I/O components 642 that are included in a particular machine will depend on the type of machine. For example, portable machines such as mobile phones will likely include a touch input device or other such input mechanisms, while a headless server machine will likely not include such a touch input device. It will be appreciated that the I/O components 642 may include many other components that are not shown in FIG. 6. The I/O components 642 are grouped according to functionality merely for simplifying the following discussion and the grouping is in no way limiting. In various example embodiments, the I/O components 642 may include output components 628 and input components 630. The output components 628 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 630 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 642 may include biometric components 632, motion components 634, environmental components 636, or position components 638, among a wide array of other components. For example, the biometric components 632 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 634 may include acceleration sensor components (e.g., accelerometer), gravitation sensor components, rotation sensor components (e.g., gyroscope), and so forth. The environmental components 636 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 638 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 642 may include communication components 640 operable to couple the machine 600 to a network 620 or devices 622 via a coupling 624 and a coupling 626, respectively. For example, the communication components 640 may include a network interface component or another suitable device to interface with the network 620. In further examples, the communication components 640 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 622 may be another machine or any of a wide variety of peripheral devices (e.g., a peripheral device coupled via a USB).

Moreover, the communication components 640 may detect identifiers or include components operable to detect identifiers. For example, the communication components 640 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, DataGlyph, MaxiCode, PDF417, Ultra Code, UCC RSS-2D bar code, and other optical codes), or acoustic detection components (e.g., microphones to identify tagged audio signals). In addition, a variety of information may be derived via the communication components 640, such as location via Internet Protocol (IP) geolocation, location via Wi-Fi® signal triangulation, location via detecting an NFC beacon signal that may indicate a particular location, and so forth.

The various memories (i.e., memory 604, main memory 612, static memory 614, and/or memory of the processors 602) and/or storage unit 616 may store one or more sets of instructions and data structures (e.g., software) embodying or utilized by any one or more of the methodologies or functions described herein. These instructions (e.g., the instructions 608), when executed by processors 602, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 620 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 620 or a portion of the network 620 may include a wireless or cellular network, and the coupling 624 may be a Code Division Multiple Access (CDMA) connection, a Global System for Mobile communications (GSM) connection, or another type of cellular or wireless coupling. In this example, the coupling 624 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 608 may be transmitted or received over the network 620 using a transmission medium via a network interface device (e.g., a network interface component included in the communication components 640) and utilizing any one of a number of well-known transfer protocols (e.g., hypertext transfer protocol (HTTP)). Similarly, the instructions 608 may be transmitted or received using a transmission medium via the coupling 626 (e.g., a peer-to-peer coupling) to the devices 622. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 608 for execution by the machine 600, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An endoscopic surgical device, comprising:
a laser source;
a laser fiber optically coupled to the laser source, the laser fiber comprising an optical core and a fiber tip, the optical core comprising a plurality of grating patterns forming a diffraction grating; and
an endoscopic probe housing an imager and the fiber tip.

2. The device of claim 1, wherein the laser source is a high-energy pulsed laser.

3. The device of claim 1, wherein the plurality of grating patterns are spaced evenly along a length of the optical core.

4. The device of claim 3, wherein the grating patterns are spaced at intervals of between 1.9 and 35 microns along the optical core.

5. The device of claim 4, wherein the intervals are between 7.5 and 7.9 microns.

6. The device of claim 1, wherein the optical core has a diameter of between 200 and 550 microns.

7. The device of claim 1, wherein each of the plurality of grating patterns is inscribed on the optical core by mechanical, chemical, or laser etching.

8. The device of claim 1, wherein each of the plurality of grating patterns is inserted into the optical core by implantation or material compositing.

9. The device of claim 1, wherein the diffraction grating is disposed within the fiber tip.

10. The device of claim 1, wherein a distal end surface of the fiber tip is inscribed with one of the plurality of grating patterns.

11. The device of claim 1, further comprising:
a display device configured to display imaging data received from the imager while the endoscopic probe is deployed.

12. The device of claim 1, further comprising:
a controller configured to, while the endoscopic probe is deployed:
receive imaging data from the imager; and
activate the laser source to discharge laser energy through the laser fiber, the laser energy emitted from the fiber tip.

13. The device of claim 12, wherein the laser energy is dispersed over an area exceeding ten times the diameter of the laser tip.

14. The device of claim 12, wherein the laser energy is dispersed over an area that includes a plurality of regions of high intensity separated by regions of lower intensity.

15. A method of endoscopic lithotripsy, comprising:
deploying an endoscopic surgical device within the urinary tract of a patient, the endoscopic surgical device comprising:
a laser source,
a laser fiber optically coupled to the laser source, the laser fiber comprising an optical core and a fiber tip, the optical core comprising a plurality of grating patterns forming a diffraction grating, and
an endoscopic probe housing an imager and the fiber tip; and
while the device is deployed, discharging laser energy from the laser source through the laser fiber to ablate a portion of a renal calculus.

16. The method of claim 15, wherein the laser source is a high-pulse energy laser.

17. The method of claim 15, wherein each of the plurality of grating patterns is inscribed on the optical core by mechanical, chemical, or laser etching.

18. The method of claim 15, wherein the diffraction grating is disposed within the fiber tip.

19. The method of claim 15, wherein a distal end surface of the fiber tip is inscribed with one of the plurality of grating patterns.

20. The method of claim 15, wherein the laser energy is dispersed over an area exceeding ten times a diameter of the laser tip.
